(19) United States
(12) Patent Application Publication
(10) Pub. No.: US 2009/0042906 A1
(43) Pub. Date: Feb. 12, 2009

(54) METHODS FOR TREATING CANCERS ASSOCIATED WITH CONSTITUTIVE EGFR SIGNALING

(75) Inventors: Hua Ming Paul Huang, Cambridge, MA (US); Forest M. White, Sutton, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/110,275

(22) Filed: Apr. 25, 2008

Related U.S. Application Data
(60) Provisional application No. 60/926,808, filed on Apr. 26, 2007.
(60) Provisional application No. 60/931,021, filed on May 16, 2007.

(51) Int. Cl. A61K 31/496; G01N 33/574; A61P 35/04

(52) U.S. Cl. 514/254.09; 435/7.23

(57) ABSTRACT

Aspects of the invention relate to methods and compositions for treating cancers associated with constitutive EGFR signaling. Methods include inhibiting one or more components of the c-Met and/or Axl signaling pathways. Aspects of the invention also relate to methods for determining whether a subject is a candidate for treatment with an inhibitor of a c-Met and/or Axl signaling component.

### RELATED APPLICATIONS

This application claims the benefit under 35 U.S.C. § 119(e) of U.S. provisional application Ser. No. 60/926,808, filed Apr. 26, 2007, and U.S. provisional application Ser. No. 60/931,021, filed May 16, 2007, the disclosures of which are incorporated by reference herein in their entirety.

### GOVERNMENT INTEREST

This work was funded in part by the NIH/NCI under grant numbers U54-CA112967, P50-GM68762 and P01-CA95616. The government has certain rights in this invention.

### FIELD OF THE INVENTION

The invention relates to methods for treating cancers associated with constitutive EGFR signaling.

### BACKGROUND OF THE INVENTION

Epidermal growth factor receptor EGFR/HER1, a member of the HER family of trans-membrane receptor tyrosine kinases, is an important mediator of cellular signal transduction. EGFR has become a target in anti-cancer therapy because it is frequently either mutated or overexpressed in cancer wherein it enhances proliferation, invasiveness angiogenesis and metastases. Several common EGFR deletion mutations have been identified in cancers including EGFRvIII, which contains an in-frame deletion of exons 2-7, resulting in a protein that lacks an extracellular ligand-binding domain. EGFRvIII, which is constitutively activated, is an attractive anti-cancer target because it is exclusive to cancers and because of its high prevalence; 60-70% of EGFR mutations in glioblastoma multiforme correspond to EGFRvIII. However, EGFRvIII-positive cancers are difficult to target, partly because they are frequently resistant to EGFR kinase inhibitors, leading to a poor prognosis for patients with these cancers. An important goal in cancer treatment is to identify methods of targeting cancers that are associated with constitutive EGFR signaling, including those expressing EGFRvIII. Another outstanding goal in cancer treatment is to be able to identify the molecular basis of a patient's cancer and based on that information be able to identify which patients would be likely to respond to a specific therapeutic approach.

### SUMMARY OF THE INVENTION

Aspects of the invention relate to methods and compositions for treating cancers associated with constitutive EGFR signaling. In some embodiments, methods include inhibiting one or more components of the c-Met signaling pathway. In certain embodiments, methods include inhibiting one or more components of the Axl signaling pathway. In some embodiments, methods include inhibiting one or more components of the c-Met signaling pathway and/or the Axl signaling pathway. In some embodiments, combinations including one or more inhibitors of the c-Met signaling pathway and/or the Axl signaling pathway along with one or more EGFR inhibitors and/or one or more chemotherapeutic agents (e.g., as a kit, a combination therapy, a recommended treatment, or any combination thereof) may be provided (e.g., prescribed and/or administered). Aspects of the invention also relate to methods for determining whether a subject is a candidate for treatment with an inhibitor of a c-Met and/or Axl signaling component.

According to one aspect of the invention, a method for treating a cancer associated with constitutive EGFR signaling is provided. The method comprises administering to a subject having a cancer that exhibits constitutive EGFR signaling a therapeutically effective amount of a composition that inhibits a c-Met and/or Axl signaling component. In certain embodiments, the method comprises administering to a subject having a cancer that exhibits constitutive EGFR signaling a composition that inhibits a c-Met signaling component and a composition that inhibits an Axl signaling component, wherein the combination of both is therapeutically effective. In some embodiments, the method comprises administering to a subject having a cancer that exhibits constitutive EGFR signaling a composition that inhibits a c-Met and/or Axl signaling component and a composition that inhibits EGFR, wherein the combination of both is therapeutically effective. In some embodiments, the cancer that exhibits constitutive EGFR signaling expresses a variant form of EGFR that contains a deletion within the extracellular domain of EGFR. In some embodiments, the cancer that exhibits constitutive EGFR signaling expresses EGFRvIII. The cancer may be glioblastoma. The c-Met signaling component may be c-Met, SHP-2, PLC-gamma or any other c-Met signaling component. The Axl signaling component may be Axl, SHP-2, PLC-gamma or any other Axl signaling component.

In some embodiments, a composition that inhibits a c-Met and/or Axl signaling component comprises a kinase inhibitor. In some embodiments, a composition that inhibits a c-Met signaling component comprises a c-Met specific kinase inhibitor. In some embodiments the c-Met specific kinase inhibitor may be SU11274. In some embodiments a composition that inhibits an Axl signaling component comprises an Axl specific kinase inhibitor (e.g., a single compound that inhibits both c-Met and Axl, both EGFR and c-Met, both EGFR and Axl, EGFR, c-Met, and Axl, or any combination including a target downstream from c-Met and Axl). In some embodiments a composition that inhibits a c-Met and/or Axl signaling component comprises a multi-target kinase inhibitor. In some embodiments a composition that inhibits a c-Met and/or Axl signaling component inhibits one or more c-Met signaling components and one or more Axl signaling components.

A composition that inhibits a c-Met or Axl signaling component may knock down expression of c-Met and/or Axl, or may comprise an antisense RNA, an RNAi, a ribozyme, or any combination thereof. In some embodiments, the composition that inhibits a c-Met and/or Axl signaling component comprises an antibody, a small molecule, a peptide, an aptamer or any combination thereof.

In some embodiments, a composition that inhibits a c-Met and/or Axl signaling component inhibits two or more c-Met and/or Axl signaling components. In some embodiments, the composition that inhibits a c-Met and/or Axl signaling component inhibits two to five c-Met and/or Axl signaling components. In some embodiments, the composition that inhibits a c-Met and/or Axl signaling component inhibits two to twenty c-Met and/or Axl signaling components. In certain embodiments a composition that inhibits one or more c-Met and/or Axl signaling component may be combined with a composition that inhibits EGFR.

One aspect of the invention relates to treating subjects having cancers that exhibit constitutive EGFR signaling with a combination of one or more compositions that inhibit a c-Met and/or Axl signaling component and a composition that inhibits EGFR signaling. In some embodiments the composition that inhibits EGFR signaling comprises a kinase inhibitor. In one embodiment the composition that inhibits EGFR signaling is AG1478. In certain embodiments the composition that inhibits EGFR comprises a multi-target inhibitor. In some embodiments the composition that inhibits EGFR also inhibits c-Met and/or SHP-2 and/or PLC-gamma. In other embodiments the composition that inhibits EGFR also inhibits Axl and/or SHP-2 and/or PLC-gamma. In other embodiments the composition that inhibits EGFR also inhibits one or more of any signaling component in the c-Met and/or Axl signaling pathways. In some embodiments, the composition that inhibits EGFR signaling knocks down expression of EGFR. In some embodiments the composition that inhibits EGFR signaling comprises an antisense RNA, an RNAi, a ribozyme, or any combination thereof. In some embodiments the composition that inhibits EGFR signaling comprises an antibody, a small molecule, a peptide, an aptamer or any combination thereof. In some embodiments, a composition that inhibits EGFR signaling comprises a dominant negative mutant form of EGFR. Similarly, in some embodiments, a composition that inhibits c-Met, Axl, or any other signaling component comprises a dominant negative form of that signaling component. It also should be appreciated that an inhibitor may be a small molecule.

According to another aspect of the invention, a method for determining whether a cancer patient should be treated with a composition that inhibits a c-Met and/or Axl signaling component is provided. The method comprises performing an assay to determine whether a patient has a cancer that exhibits constitutive EGFR signaling and identifying the patient as being a candidate for treatment with a composition that inhibits a c-Met and/or Axl signaling component if the patient has a cancer that expresses c-Met and/or Axl and exhibits constitutive EGFR signaling (e.g., notifying the patient and/or the patient's physician or health care provider, including a diagnosis in the patient's medical record, recommending or prescribing a therapy or course of treatment, etc., or any combination thereof). In some embodiments, the cancer that exhibits constitutive EGFR signaling expresses a variant form of EGFR that contains a deletion within the extracellular domain of EGFR. In some embodiments, the cancer that exhibits constitutive EGFR signaling expresses EGFRvIII. The cancer may be glioblastoma. The c-Met signaling component may be c-Met, SHP-2 or PLC-gamma or any other c-Met signaling component. The Axl signaling component may be Axl, SHP-2 or PLC-gamma or any other Axl signaling component.

In some embodiments, a patient is prescribed or treated with one or more compositions of the invention based on a diagnosis or knowledge of the presence of condition (e.g., cancer) characterized by the presence of constitutive EGFR signaling (e.g., in the presence of c-Met and/or Axl expression). In some embodiments, a subject is tested for the presence of an PTEN mutation and a therapy of the invention may be administered or recommended, at least in part, based on the presence of a PTEN mutation in addition to constitutive EGFR signaling.

In some embodiments, the composition that inhibits a c-Met and/or Axl signaling component comprises a kinase inhibitor. In some embodiments, the composition that inhibits a c-Met signaling component comprises a c-Met specific kinase inhibitor. In some embodiments the c-Met specific kinase inhibitor may be SU11274. In some embodiments, the composition that inhibits an Axl signaling component comprises an Axl specific kinase inhibitor.

The composition that inhibits a c-Met and/or Axl signaling component may knock down expression of c-Met and/or Axl, or may comprise an antisense RNA, an RNAi, a ribozyme, or any combination thereof. In some embodiments, the composition that inhibits a c-Met and/or Axl signaling component comprises an antibody, a small molecule, a peptide, an aptamer or any combination thereof. In some embodiments, the composition that inhibits a c-Met and/or Axl signaling component comprises a dominant negative mutant form of c-Met and/or Axl.

In some embodiments, the composition that inhibits a c-Met and/or Axl signaling component inhibits two or more c-Met and/or Axl signaling components. In some embodiments, the composition that inhibits a c-Met and/or Axl signaling component inhibits two to five c-Met and/or Axl signaling components. In some embodiments, the composition that inhibits a c-Met and/or Axl signaling component inhibits two to twenty c-Met and/or Axl signaling components.

In some embodiments, the act of determining whether a patient has a cancer that exhibits constitutive EGFR signaling comprises assaying for constitutive EGFR signaling by a kinase assay.

In some embodiments, the act of determining whether a patient has a cancer that exhibits constitutive EGFR signaling comprises assaying for a variant form of EGFR. The variant form of EGFR may be assayed for by Western Blot analysis or by ELISA. The variant form of EGFR may also be assayed for by sequencing the EGFR gene or by Northern Blot analysis.

Aspects of the invention relate to compositions and methods for decreasing the growth and/or viability of a cell (e.g., a cancer cell) that exhibits constitutive EGFR signaling. In some embodiments, a cell that exhibits constitutive EGFR signaling with is contacted with a composition that inhibits a c-Met signaling component in an amount effective to decrease the proliferation and/or viability of the cancer cell. In some embodiments, methods include contacting a cell (e.g., a cancer cell) that exhibits constitutive EGFR signaling with a combination of a composition that inhibits a c-Met signaling component and a composition that inhibits EGFR signaling each in an amount sufficient for the combination to decrease the proliferation and/or viability of the cell. In some embodiments, the methods include contacting a cell (e.g., a cancer cell) that exhibits constitutive EGFR signaling with a composition that inhibits an Axl signaling component in an amount effective to decrease the proliferation and/or viability of the cell. In some embodiments, methods include contacting a cell (e.g., a cancer cell) that exhibits constitutive EGFR signaling with a combination of a composition that inhibits an Axl signaling component and a composition that inhibits EGFR signaling each in an amount sufficient for the combination to decrease the proliferation and/or viability of the cell.

It should be appreciated that a cell that exhibits constitutive EGFR signaling may be identified as a cell that contains an EGFR variant or mutant form known to be associated with constitutive EGFR signaling. According to some embodiments, a cell that exhibits constitutive EGFR signaling may be identified as a cell that has high levels of c-MET/Axl phosphorylation in addition to the presence of a mutation in EGFR relative to wild-type EGFR. In some embodiments, high levels of EGFR expression (e.g., relative to wild-type, e.g., about 1-2 fold, about 2-4 fold, about 4-8 fold, about 8-20 fold, about 20-50 fold, or higher levels of expression relative to wild-type) are sufficient for constitutive EGFR signaling. It should be appreciated that expression may be measured as an RNA level and/or a protein level.

These and other aspects of the invention are described in more detail herein.

### BRIEF DESCRIPTION OF THE DRAWINGS

FIG. 1 illustrates examples of cell lines and a non-limiting experimental strategy —

FIG. 1A is a table indicating EGFRvIII expression levels in retrovirally transfected U87MG cell lines, FIG. 1B is a Western blot of U87MG cell lines expressing titrated levels of EGFRvIII, and FIG. 1C is a schematic showing an outline of an MS-based experimental strategy;

FIG. 2 shows the effect of EGFRvIII receptor levels on downstream signaling networks—FIG. 2A is a graph showing relative quantification of EGFRvIII phosphorylation sites across the four cell lines, and FIG. 2B is a schematic showing the fold change in phosphorylation levels in the canonical EGFR signaling cascade as a function of titrated EGFRvIII levels;

FIG. 3 shows activation of signaling networks downstream of EGFRvIII—FIG. 3A shows a clustering analysis of phosphotyrosine protein networks using self-organizing maps (SOMs), FIG. 3B shows protein phosphorylation sites found within a highly responsive cluster, FIG. 3C is a Western blot showing specific phosphorylation sites on the c-Met receptor (Y1230/Y1234/Y1235) across the four different cell lines in vitro after 24-h serum starvation, and FIG. 3D is a Western blot showing c-Met receptor phosphorylation levels of in vivo parental (P), DK, or EGFRvIII high-expressing U87MG-derived xenografts;

FIG. 4 shows c-Met receptor activation and kinase inhibition—FIG. 4A is a Western blot of U87-H cells subjected to 1 h AG1478 dose escalation after 24-h serum starvation, FIG. 4B is a Western blot showing U87-H cells subjected to dose escalation of SU11274, and FIG. 4C shows a comparison of the quantification of the phosphorylation levels for c-Met Y1234 upon treatment with either DMSO (control) or 10 μM c-Met kinase inhibitor SU11274 for 1 h after 24-h serum starvation;

FIG. 5 shows a dose-response of the U87-H cell line upon treatment with kinase inhibitors or cisplatin—FIG. 5A is a graph showing a dose-response of U87-H cells to AG1478, SU1127, or a combination of SU11274 and 5 μM AG1478 over 72 h after 24-h serum starvation, FIG. 5B is a graph showing apoptosis measured by caspase 3/7 cleavage upon drug treatment over 24 h after 24-h serum starvation, FIG. 5C is a graph showing a dose-response of U87-H cells to AG1478, PHA665752, or a combination of PHA665752 and 5 μM AG1478 over 72 h after 24-h serum starvation, and FIG. 5D is a graph showing the viability of U87-H cells in response to a combination treatment of 10 g/ml cisplatin with either AG1478 or SU11274;

FIG. 6 shows EGFRvIII levels expressed in engineered U87MG cells—FIGS. 6A-E illustrate relative levels of membrane-expressed receptors in different cell lines as determined by FITC-conjugated antibody staining fluorescence intensity, and FIG. 6F summarizes the data;

FIG. 7 shows activation of c-Met receptor by EGFRvIII observed in U373MG cells;

FIG. 8 is a schematic showing activation of the c-Met receptor network by EGFRvIII;

FIG. 9 shows that activation of the c-Met receptor by EGFRvIII is ligand-independent—FIG. 9A is a graph showing measurement of HGF secreted into the media after 24-h serum starvation, and FIG. 9B is a Western blot showing specific phosphorylation sites on the c-Met receptor (Y1230, Y1234, and Y1235);

FIG. 10 is a graph showing that U87H cells are resistant to treatment with cisplatin; and,

FIG. 11 shows activation of signaling networks downstream of EGFRvIII—FIG. 11A shows clustering analysis of phosphotyrosine protein networks using self-organizing maps (SOMs), and FIG. 11B shows protein phosphorylation of Axl receptor Y693.

### DETAILED DESCRIPTION

Aspects of the invention relate to methods and compositions for treating cancers associated with constitutive EGFR signaling. The invention relates at least in part to the finding that the c-Met and Axl receptors are phosphorylated in glioblastoma cell lines in response to expression of a variant form of EGFR, EGFRvIII, that produces constitutive EGFR signaling. It has previously been observed that cancer cells that express EGFRvIII exhibit resistance to EGFR inhibitors when the function of an additional gene, phosphatase and tensin homologue deleted on chromosome 10 (PTEN) is lost, a common occurrence in glioblastomas. Aspects of the invention relate at least in part to the finding that treatment of EGFRvIII-expressing cell lines with compositions that inhibit components of the c-Met signaling pathway either alone or in combination with EGFR inhibitors, leads to a dose-dependent decrease in cell growth and increase in apoptosis. Significantly, this treatment is effective even when the function of the PTEN gene is lost. The invention provides methods for using compositions that inhibit components of the c-Met and/or Axl signaling pathways, either alone or in combination with EGFR inhibitors or chemotherapeutic agents, to target cancers that exhibit constitutive EGFR signaling. The invention further provides a method for determining whether a cancer patient should be treated with a composition that includes one or more compositions that inhibit a c-Met signaling component and/or an Axl signaling component and/or EGFR based on the determination of whether a patient has a cancer that is associated with constitutive EGFR signaling.

Aspects of the invention relate to cancers that exhibit constitutive EGFR signaling. These may include cancers that express any mutation in EGFR that causes it to be constitutively active. In some embodiments, a cancer associated with constitutive EGFR signaling may express a mutated form of EGFR in which there is a deletion within the extracellular domain. In certain embodiments, a mutated form of EGFR is EGFRvIII. In some embodiments, a mutation causing EGFR constitutive signaling may be caused by a point mutation, deletion, insertion, duplication, inversion or any other mutation, or any combination thereof, in the extracellular domain of EGFR (e.g., in the portion of the EGFR gene encoding the extracellular domain) that gives rise to constitutive EGFR signaling. In certain embodiments, a mutation may be a mutation in the intracellular domain of EGFR (e.g., a deletion, point mutation, insertion, duplication, inversion, etc., or any combination thereof) that leads to constitutive EGFR signaling.

It should be appreciated that constitutive EGFR signaling may be detected using any suitable direct or indirect assay for detecting a constitutively active EGFR variant in a patient sample. In some embodiments, constitutive EGFR signaling may be detected using a kinase assay (e.g., an EGFR specific kinase assay). In certain embodiments, constitutive EGFR signaling may be detected by a Western blot (e.g., with a phospho-specific antibody) to detect phosphorylated EGFR, or by an ELISA assay. In some embodiments, constitutive EGFR signaling may be inferred from the detection of a mutated form of EGFR that is known to cause constitutive EGFR signaling. The means of identifying mutated forms of EGFR could be by Northern Blot analysis or by PCR amplification of the locus and sequencing of the locus to look for mutations (e.g., a deletion of one or more exon encoding sequences, e.g., a deletion of one or more of exons 2-7 of EGFR).

It should be appreciated that a constitutively active EGFR as used herein relates to EGFR activation that is ligand independent (e.g., independent of activation by a ligand from the EGF family of ligands). However, according to aspects of the invention, a constitutively active EGFR variant may have a constitutive (ligand independent) level of activation (e.g., as measured by the level of EGFR phosphorylation) that is different from the level of activation of wild-type (e.g., normal ligand-dependent) EGFR activation in response to a ligand. For example, a constitutively active variant of EGFR may have a constitutive activation level of between 1% and 100% of wild type activation in response to a ligand (e.g., between 5% and 95%, at least 10%, at least 20%, at least 30%, at least 40%, at least 50%, at least 60%, at least 70%, at least 80%, at least 90% of the wild-type activated level). In some embodiments, a constitutively active variant of EGFR may have a constitutive activation level that is greater than 100% of wild type activation in response to a ligand (e.g., 2 fold, 3 fold, 4 fold etc.) However, aspects of the invention also may include variants with lower or higher constitutively active levels. In some embodiments, EGFRvIII has a constitutive level of activation that is about 10% of the activated wild-type level of activation.

In some embodiments, constitutive activation results from over-expression of EGFR. However, in some embodiments a phenotype associated with constitutive EGFR activity may result from a mutant EGFR receptor that is constitutively active. Accordingly, methods and compositions of the invention may be used to treat cancers associated with constitutively active EGFR receptors. It should be appreciated that aspects of the invention relate to treating cancers that are characterized by constitutive EGFR expression (e.g., in the presence of EGFRvIII) that activates one or more components of the c-Met and/or Axl signaling pathways. Accordingly, aspects of the invention relate to treatments for cancers that are known to express c-Met and/or Axl. According to the invention, c-Met and/or Axl does not need to be over-expressed (e.g., normal c-Met and/or Axl levels may be observed) for treatment to be recommended, prescribed, and/or administered. However, c-Met and/or Axl under-expression or over-expression may be acceptable. In some embodiments, expression of a mutated form of c-Met and/or Axl may be acceptable. Accordingly, some aspects of the invention include assaying for c-Met and/or Axl expression in addition to assaying for constitutive EGFR signaling. However, if certain cells, tissues, or cancers, are known to express c-Met and/or Axl, (e.g., in glioblastomas), then assays for constitutive EGFR signaling alone may be sufficient.

It should be appreciated that aspects of the invention relate to treating cancers that are characterized by constitutive EGFR signaling (e.g., in the presence of EGFRvIII) regardless of the status of the PTEN gene. In some embodiments the cancer will exhibit constitutive EGFR signaling, express c-Met and/or Axl and express the PTEN gene. In other embodiments, the cancer will exhibit constitutive EGFR signaling, express c-Met and/or Axl and not express the PTEN gene. In some embodiments, the cancer will exhibit constitutive EGFR signaling, express c-Met and/or Axl, and express a mutated form of the PTEN gene. Some aspects of the invention include assaying for PTEN (e.g., PTEN mutations or PTEN underexpression) in addition to assaying for constitutive EGFR signaling.

It should be appreciated that c-Met and/or Axl expression and/or signaling activity may be detected using any suitable direct or indirect assay for detecting c-Met and/or Axl expression and/or signaling activity in a patient sample. In some embodiments, c-Met and/or Axl signaling activity may be detected using a kinase assay (e.g., a c-Met and/or Axl specific kinase assay). In certain embodiments, c-Met and/or Axl signaling activity may be detected by a Western Blot (e.g., with a phospho-specific antibody), or by an ELISA assay. In some embodiments, c-Met and/or Axl signaling activity may be inferred from the detection of the c-Met and/or Axl mRNAs. The means of identifying c-Met and/or Axl mRNA could be by Northern Blot analysis. In some embodiments, a mutated form of the c-Met and/or Axl genes may be detected. The means of identifying a mutated form of c-Met and/or Axl could be by Northern Blot analysis or by PCR amplification of the locus and sequencing of the locus to look for mutations (e.g., a deletion of one or more exon encoding sequences). It should be appreciated that PTEN expression may be detected using any suitable direct or indirect assay for detecting PTEN expression in a patient sample. In some embodiments, a mutated form of the PTEN gene may be detected. The means of identifying PTEN mRNA expression may be by Northern blot analysis or by PCR amplification of the locus and sequencing of the locus. The means of detecting PTEN protein expression may be by Western blot analysis. The means of identifying a mutated form of PTEN could be by Northern blot analysis or by PCR amplification of the locus and sequencing of the locus to look for mutations (e.g., a deletion of one or more exon encoding sequences).

An assay for detecting the presence of a constitutively active EGFR variant and/or for c-Met and/or Axl and/or PTEN expression as described herein may be performed on any suitable tissue biopsy (e.g., cancer tissue biopsy) or other suitable biological sample (e.g., blood, serum, urine, sputum, stool, CSF, or any other biological fluid, or any combination thereof).

According to some aspects of the invention, a subject (e.g., a cancer patient) may be identified as a candidate for treatment with a composition that inhibits a c-Met and/or Axl signaling component if the subject has a disease (e.g., a cancer) that expresses a constitutively active variant of EGFR (e.g., EGFRvIII) in at least some, if not all, of the cancer cells. Accordingly, in some embodiments a subject (e.g., a cancer patient) is tested for the presence of a constitutively active EGFR variant, and if present, is identified as a candidate for treatment with a composition that inhibits a c-Met and/or Axl signaling component either alone or in combination with EGFR inhibitors. In some embodiments a subject (e.g., a cancer patient) is tested for the presence of a constitutively active EGFR variant, and for the expression of c-Met and/or Axl, and if a constitutively active EGFR variant is detected, and c-Met and/or Axl expression is detected, then the subject is identified as a candidate for treatment with a composition that inhibits a c-Met and/or Axl signaling component either alone or in combination with EGFR inhibitors. In some embodiments, a subject (e.g., a cancer patient) is tested for the presence of a constitutively active EGFR variant, the expression of c-Met and/or Axl, and the expression of PTEN, and if a constitutively active EGFR variant is detected, c-Met and/or Axl expression is detected, and no PTEN expression is detected (or expression of a PTEN mutant is detected), then the subject is identified as a candidate for treatment with a composition that inhibits a c-Met and/or Axl signaling component either alone or in combination with EGFR inhibitors. In certain embodiments a subject (e.g., a cancer patient) is tested for the presence of a constitutively active EGFR variant, the expression of c-Met and/or Axl, and the expression of PTEN, and if a constitutively active EGFR variant is detected, c-Met and/or Axl expression is detected, and PTEN expression is detected, then the subject is identified as a candidate for treatment with a composition that inhibits a c-Met and/or Axl signaling component either alone or in combination with EGFR inhibitors.

In some embodiments, a subject (e.g., a cancer patient) who has a disease (e.g., a cancer) that expresses a constitutively active variant of EGFR (e.g., EGFRvIII) in at least some, if not all, of the cancer cells, and who is identified as a candidate for treatment with a composition that inhibits a c-Met and/or Axl signaling component, may be recommended or prescribed a treatment that includes one or more compounds that inhibit a component of the c-Met and/or Axl signaling pathway (e.g., c-Met or a downstream component of the c-Met pathway and or Axl or a downstream component of the Axl pathway).

In some embodiments, an inhibitor of EGFR (e.g., an inhibitor of EGFR activity, expression, etc., or any combination thereof) is also recommended, prescribed, or administered to the subject. In some embodiments, a chemotherapeutic agent is also recommended, prescribed, and/or administered to the subject. According to aspects of the invention, certain combinations of EGFR inhibitors and c-Met and/or Axl signaling component inhibitors may have synergistic inhibitory effects on constitutive EGFR expressing cancers (see the Examples). According to aspects of the invention, chemotherapeutic agents may be effective in the presence of c-Met and/or Axl signaling component inhibitors in otherwise chemotherapeutic resistant cancers (e.g., cancers that express constitutively active EGFR such as EGFRvIII). In some embodiments, a combination of one or more EGFR inhibitors, one or more c-Met signaling component inhibitors, one or more Axl signaling component inhibitors and/or one or more chemotherapeutic agents may be recommended, prescribed, and/or administered to a subject that has been identified as having a condition (e.g., a cancer) associated with constitutive EGFR expression.

Aspects of the invention relate to using one or more EGFR inhibitors. It should be appreciated that an EGFR inhibitor may inhibit expression (e.g., transcription, translation, and/or stability) of EGFR and/or EGFR activity. An inhibitor may be a specific EGFR inhibitor or a non-specific inhibitor (e.g., a non-specific kinase inhibitor) or a multi-target inhibitor that inhibits EGFR. An inhibitor may be a small molecule, an aptamer, an antibody, an RNAi, an antisense RNA, or any other suitable molecule, or any combination thereof. Examples of EGFR inhibitors include Erlotinib, Gefitinib, AG1478, Laptinib, and others, or any combination thereof.

Aspects of the invention relate to using one or more c-Met and/or Axl signaling component inhibitors. It should be appreciated that a c-Met and/or Axl signaling component inhibitor may inhibit expression (e.g., transcription, translation, and/or stability) and/or activity of one or more components of the c-Met and/or Axl signaling pathways (e.g., c-Met (NM_{—}000245), or a downstream component of the c-Met signaling pathway, for example SHP-2/PTPN11 (NM_{—}002834), PLC-gamma (NM_{—}002660, NM_{—}182811, NM_{—}002661), or any one or more other downstream components, or any combination of two or more thereof, Axl (NM_{—}021913, NM_{—}001699), or a downstream component of the Axl signaling pathway, for example SHP-2/PTPN11 (NM_{—}002834), PLC-gamma (NM_{—}002660, NM_{—}182811, NM_{—}002661), or any one or more other downstream components, or any combination of two or more thereof). In some embodiments a downstream component of the c-Met and/or Axl signaling pathways comprises a component of the PI3K pathway including but not limited to PI3K and Akt. In certain embodiments a downstream component of the c-Met and/or Axl signaling pathways comprises an enzymatic downstream component including but not limited to SHP-2, PLC-gamma and PI3K. In some embodiments a downstream component of the c-Met and/or Axl signaling pathways includes a structural downstream component including but not limited to SHC and GAB1. An inhibitor may be a specific inhibitor or a non-specific inhibitor (e.g., a non-specific kinase inhibitor) or a multi-target inhibitor that inhibits one or more c-Met and/or Axl signaling components. An inhibitor may be a small molecule, an aptamer, an antibody, an RNAi, an shRNA, an antisense RNA, or any other suitable molecule, or any combination thereof. An inhibitor could also comprise a composition expressing a dominant negative mutant version of c-Met and/or Axl and/or any component of the c-Met and/or Axl signaling pathways. Examples of c-Met inhibitors include SU11274, PHA665752, and others, or any combination thereof. In some embodiments, inhibitors of one or more downstream components (e.g., mTor) also may be used alone or in combination with any of the others described herein. Non-limiting examples of mTor inhibitors include Rapamycin and PI-103.

It should be appreciated that the EGFR, c-Met and/or Axl pathways may also be inhibited through inhibition of ligands that activate these signaling pathways. For example the HGF ligand for c-Met or the Gas6 ligand for Axl may be targeted. An inhibitor of a ligand may be a small molecule, an aptamer, an antibody, an RNAi, an shRNA, an antisense RNA, or any other suitable molecule, or any combination thereof. An inhibitor of a ligand may be used in combination with an inhibitor of another component of one or more of the EGFR, c-Met and/or Axl signaling pathways. A non-limiting example of an inhibitor of an EGFR ligand is Cetuximab.

Aspects of the invention relate to using one or more chemotherapeutic agents. A chemotherapeutic agent may be an alkylating agent (e.g., Temozolomide), a nucleic acid (e.g., DNA) damaging agent, or other suitable chemotherapeutic agent. In some embodiments, a chemotherapeutic agent is a platinum based compound (e.g., cisplatin or related compound).

Aspects of the invention relate to co-treatments with one or more of the inhibitors described herein. Accordingly, aspects of the invention relate to kits or compositions comprising combinations of two or more (e.g., 2, 3, 4, 5, 6, 7, 8, 9, 10, or more) inhibitors described herein. For example, one or more inhibitors of the c-Met signaling pathway may be combined with one or more inhibitors of the Axl signaling pathway. As well, one or more c-Met and/or Axl signaling component inhibitors may be combined with one or more EGFR inhibitors, and/or one or more chemotherapeutic agents. In certain embodiments one or more inhibitors of a c-Met signaling component may be combined with one or more inhibitors of PI3K and one or more inhibitors of EGFR. In certain embodiments one or more inhibitors of an Axl signaling component may be combined with one or more inhibitors of PI3K and one or more inhibitors of EGFR. In some embodiments, one or more compositions that inhibit a c-Met signaling component and/or an Axl signaling component and/or EGFR and/or a chemotherapeutic agent may be combined with radiation therapy.

In some embodiments, a single compound may inhibit one or more of EGFR, a c-Met signaling component, and/or an Axl signaling component (e.g., EGFR and c-Met, EGFR and SHP-2, EGFR and PLC-gamma, c-Met and SHP-2, c-Met and PLC-gamma, EGFR and Axl, Axl and SHP-2, Axl and PLC-gamma, c-Met and Axl, or any other combination thereof).

It should be appreciated that aspects of the invention are useful for treating cancers or other conditions associated with constitutive EGFR signaling (e.g., caused by EGFRvIII or other constitutive EGFR variant or other mutation that causes constitutive EGFR activity) in the presence of c-Met and/or Axl expression in humans or other mammals or other vertebrates. Accordingly, aspects of the invention relate to inactivating human genes or proteins described herein in human subjects. However, equivalent therapeutic techniques and compositions may be used in other mammals (e.g., domestic animals or farm animals such as dogs, cats, horses etc.).

It should be appreciated that any cancer characterized by constitutive EGFR signaling and c-Met and/or Axl expression may be treated according to aspects of the invention. For example, any suitable neural, brain, CNS, colorectal, liver, kidney, lung, pancreatic, adrenal, bone, osophageal, gastric, or other cancer (e.g., any cancer of epithelial origin) characterized by constitutive EGFR signaling and c-Met and/or Axl expression (in at least a subset of the cell within cancerous tissue) may be treated according to aspects of the invention. In some embodiments, glioblastomas (e.g., primary and/or secondary glioblastomas) may be treated according to aspects of the invention. In some embodiments, recurring or chemoresistant cancers may be treated according to aspects of the invention. In some embodiments, glioblastomas that are resistant to EGFR kinase inhibitors may be treated according to aspects of the invention. In some embodiments, glioblastomas that have lost PTEN function may be treated according to aspects of the invention. In certain embodiments glioblastomas that exhibit constitutive EGFR signaling, have lost the function of the PTEN gene and are resistant to EGFR kinase inhibitors may be treated according to aspects of the invention.

Compositions of the invention may be administered in effective amounts. An effective amount is a dosage of the composition of the invention sufficient to provide a medically desirable result. An effective amount means that amount necessary to delay the onset of, inhibit the progression of or halt altogether the onset or progression of the particular condition (e.g., constitutive EGFR-associated cancer) being treated. An effective amount may be an amount that reduces one or more signs or symptoms of the condition (e.g., constitutive EGFR-associated cancer). When administered to a subject, effective amounts will depend, of course, on the particular condition being treated (e.g., the EGFR-associated cancer), the severity of the condition, individual subject parameters including age, physical condition, size and weight, concurrent treatment, frequency of treatment, and the mode of administration. These factors are well known to those of ordinary skill in the art and can be addressed with no more than routine experimentation.

Actual dosage levels of active ingredients in the compositions of the invention can be varied to obtain an amount of the composition of the invention that is effective to achieve the desired therapeutic response for a particular subject, compositions, and mode of administration. The selected dosage level depends upon the activity of the particular composition, the route of administration, the severity of the condition being treated, the condition, and prior medical history of the subject being treated. However, it is within the skill of the art to start doses of the composition at levels lower than required to achieve the desired therapeutic effort and to gradually increase the dosage until the desired effect is achieved. In some embodiments, lower dosages would be required for combinations of multiple compositions than for single compositions (e.g., a composition that inhibits a c-Met signaling component combined with a composition that inhibits an Axl signaling component, a composition that inhibits a c-Met and/or Axl signaling component combined with a composition that inhibits EGFR, may require lower dosages when administered in combination than when administered singly). Similarly, lower dosages may be required for multi-target inhibitors that inhibit more than one of any component of the c-Met signaling pathway, and/or any component of the Axl signaling pathway, and/or EGFR, than for single-target inhibitors.

The compositions of the invention can be administered to a subject by any suitable route. For example, the compositions can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain, in the abdominal cavity, under the splenic capsule, brain, or in the cornea.

Compositions of the present invention also can be administered in the form of liposomes. As is known in the art, liposomes generally are derived from phospholipids or other lipid substances. Liposomes are formed by mono- or multi-lamellar hydrated liquid crystals that are dispersed in an aqueous medium. Any nontoxic, physiologically acceptable, and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p 33, et seq.

Dosage forms for topical administration of a composition of this invention include powders, sprays, ointments, and inhalants as described herein. The composition is mixed under sterile conditions with a pharmaceutically acceptable carrier and any needed preservatives, buffers, or propellants which may be required. Ophthalmic formulations, eye ointments, powders, and solutions also are contemplated as being within the scope of this invention.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water ethanol, polyols (such as, glycerol, propylene glycol, polyethylene glycol, and the like), and suitable mixtures thereof, vegetable oils (such, as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents which delay absorption, such as aluminum monostearate and gelatin.

In some cases, in order to prolong the effect of the composition, it is desirable to slow the absorption of the composition from subcutaneous or intramuscular injection. This result can be accomplished by the use of a liquid suspension of crystalline or amorphous materials with poor water solubility. The rate of absorption of the composition then depends upon its rate of dissolution which, in turn, may depend upon crystal size and crystalline form. Alternatively, delayed absorption of a parenterally administered composition from is accomplished by dissolving or suspending the composition in an oil vehicle.

Injectable depot forms are made by forming microencapsule matrices of the composition in biodegradable polymers such a polylactide-polyglycolide. Depending upon the ratio of composition to polymer and the nature of the particular polymer employed, the rate of composition release can be controlled. Examples of other biodegradable polymers include poly(orthoesters) and poly(anhydrides). Depot injectable formulations also are prepared by entrapping the drug in liposomes or microemulsions which are compatible with body tissue.

The injectable formulations can be sterilized, for example, by filtration through a bacterial- or viral-retaining filter, or by incorporating sterilizing agents in the form of sterile solid compositions which can be dissolved or dispersed in sterile water or other sterile injectable medium just prior to use.

The invention provides methods for oral administration of a pharmaceutical composition of the invention. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation includes a composition of the invention and inert ingredients which protect against degradation in the stomach and which permit release of the biologically active material in the intestine.

In such solid dosage forms, the composition is mixed with, or chemically modified to include, a least one inert, pharmaceutically acceptable excipient or carrier. The excipient or carrier preferably permits (a) inhibition of proteolysis, and (b) uptake into the blood stream from the stomach or intestine. In one embodiment, the excipient or carrier increases uptake of the composition of the invention, overall stability of the composition and/or circulation time of the composition in the body. Excipients and carriers include, for example, sodium citrate or dicalcium phosphate and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropylmethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffin, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

Solid compositions of a similar type also can be employed as fillers in soft and hard-filled gelatin capsules, using such excipients as lactose or milk sugar, as well as high molecular weight polyethylene glycols and the like.

The solid dosage forms of tablets, dragees, capsules, pills, and granules can be prepared with coatings and shells, such as enteric coatings and other coatings well known in the pharmaceutical formulating art. They optionally can contain opacifying agents and also can be of a composition that they release the active ingredients(s) only, or preferentially, in a part of the intestinal tract, optionally, in a delayed manner. Exemplary materials include polymers having pH sensitive solubility, such as the materials available as EUDRAGIT® Examples of embedding compositions which can be used include polymeric substances and waxes.

The composition of the invention also can be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the composition of the invention, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydroflirfuryl alcohol, polyethylene glycols, fatty acid esters of sorbitan, and mixtures thereof.

Besides inert diluents, the oral compositions also can include adjuvants, such as wetting agents, emulsifying and suspending agents, sweetening, coloring, flavoring, and perfuming agents. Oral compositions can be formulated and further contain an edible product, such as a beverage.

Suspensions, in addition to the composition of the invention, can contain suspending agents such as, for example ethoxylated isostearyl alcohols, polyoxyethylene sorbitol and sorbitan esters, microcrystalline cellulose, aluminum metahydroxide, bentonite, agar-agar, tragacanth, and mixtures thereof.

Also contemplated herein is pulmonary delivery of the composition of the invention. The composition is delivered to the lungs of a mammal while inhaling, thereby promoting the traversal of the lung epithelial lining to the blood stream. See, Adjei et al., Pharmaceutical Research 7:565-569 (1990); Adjei et al., International Journal of Pharmaceutics 63:135-144 (1990) (leuprolide acetate); Braquet et al., Journal of Cardiovascular Pharmacology 13 (suppl.5): s.143-146 (1989)(endothelin-1); Hubbard et al., Annals of Internal Medicine 3:206-212 (1989)(α1-antitrypsin); Smith et al., J. Clin. Invest. 84:1145-1146 (1989) (α1-proteinase); Oswein et al., “Aerosolization of Proteins,” Proceedings of Symposium on Respiratory Drug Delivery II, Keystone, Colo., March, 1990 (recombinant human growth hormone); Debs et al., The Journal of Immunology 140:3482-3488 (1988) (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including, but not limited to, nebulizers, metered dose inhalers, and powder inhalers, all of which are familiar to those skilled in the art.

Some specific examples of commercially available devices suitable for the practice of the invention are the ULTRAVENT® nebulizer, manufactured by Mallinckrodt, Inc., St. Louis, Mo.; the ACORN II® nebulizer, manufactured by Marquest Medical Products, Englewood, Colo.; the VENTOL® metered dose inhaler, manufactured by Glaxo Inc., Research Triangle Park, N.C.; and the SPINHALER® powder inhaler, manufactured by Fisons Corp., Bedford, Mass.

All such devices require the use of formulations suitable for the dispensing of a composition of the invention. Typically, each formulation is specific to the type of device employed and can involve the use of an appropriate propellant material, in addition to diluents, adjuvants, and/or carriers useful in therapy.

In some embodiments, the composition is prepared in particulate form, preferably with an average particle size of less than 10 μm, and most preferably 0.5 to 5 μm, for most effective delivery to the distal lung.

Carriers include carbohydrates such as trehalose, mannitol, xylitol, sucrose, lactose, and sorbitol. Other ingredients for use in formulations may include lipids, such as DPPC, DOPE, DSPC and DOPC, natural or synthetic surfactants, polyethylene glycol (even apart from its use in derivatizing the inhibitor itself), dextrans, such as cyclodextran, bile salts, and other related enhancers, cellulose and cellulose derivatives, and amino acids.

Also, the use of liposomes, microcapsules or microspheres, inclusion complexes, or other types of carriers is contemplated.

Formulations suitable for use with a nebulizer, either jet or ultrasonic, typically comprise a composition of the invention dissolved in water at a concentration of about 0.1 to 25 mg of biologically active protein per mL of solution. The formulation also can include a buffer and a simple sugar (e.g., for protein stabilization and regulation of osmotic pressure). The nebulizer formulation also can contain a surfactant to reduce or prevent surface-induced aggregation of the inhibitor composition caused by atomization of the solution in forming the aerosol.

Formulations for use with a metered-dose inhaler device generally comprise a finely divided powder containing the composition of the invention suspended in a propellant with the aid of a surfactant. The propellant can be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid also can be useful as a surfactant.

Formulations for dispensing from a powder inhaler device comprise a finely divided dry powder containing the composition of the invention and also can include a bulking agent, such as lactose, sorbitol, sucrose, mannitol, trehalose, or xylitol, in amounts which facilitate dispersal of the powder from the device, e.g., 50 to 90% by weight of the formulation.

Nasal delivery of the composition of the invention also is contemplated. Nasal delivery allows the passage of the composition to the blood stream directly after administering the therapeutic product to the nose, without the necessity for deposition of the product in the lung. Formulations for nasal delivery include those with dextran or cyclodextran. Delivery via transport across other mucous membranes also is contemplated.

Compositions for rectal or vaginal administration are preferably suppositories which can be prepared by mixing the composition of the invention with suitable nonirritating excipients or carriers, such as cocoa butter, polyethylene glycol, or suppository wax, which are solid at room temperature, but liquid at body temperature, and therefore melt in the rectum or vaginal cavity and release the active compound.

In order to facilitate delivery of the composition of the invention across cell and/or nuclear membranes, compositions of relatively high hybrophobicity are preferred. The composition of the invention can be modified in a manner which increases hydrophobicity, or the composition of the invention can be encapsulated in hydrophobic carriers or solutions which result in increased hydrophobicity.

It should be appreciated that any compositions of the invention described herein may be sterilized (e.g., for storage and/or prior to administration to a subject) and may be provided in a physiologically acceptable formulation (e.g., along with one or more physiologically acceptable buffers, salts, and/or other components).

The term “treatment” or “treating” is intended to relate to prophylaxis, amelioration, prevention and/or cure of a condition (e.g., constitutive EGFR-associated cancer). Treatment after a condition (e.g., EGFR-associated cancer) that has started aims to reduce, ameliorate or altogether eliminate the condition, and/or its associated symptoms, or prevent it from becoming worse. Treatment of subjects before a condition (e.g., EGFR-associated cancer) has started (i.e., prophylactic treatment) aims to reduce the risk of developing the condition and/or lessen its severity if the condition does develop. As used herein, the term “prevent” refers to the prophylactic treatment of a subject who is at risk of developing a condition (e.g., EGFR-associated cancer) resulting in a decrease in the probability that the subject will develop the disorder, and/or to the inhibition of further development of an already established disorder.

### EXAMPLES

Aspects of the invention are illustrated by the following non-limiting examples. It should be appreciated that these examples are non-limiting and exemplify certain aspects and embodiments of the invention described herein.

### Example 1

### Quantitative Analysis of EGFRvIII Cellular Signaling Networks Reveals a Novel Combinatorial Therapeutic Strategy in Glioblastoma

Glioblastoma multiforme (GBM) is the most aggressive form of adult human brain tumor with median survival of less than 12 months. This dismal prognosis is due in part to the lack of therapeutic agents available to eliminate the diffused glioma infiltrate that remains in the brain after surgical resection. In this study, a previously described mass spectrometry-based phosphoproteomics approach was used to quantitatively map cellular signaling events activated by the EGFRvIII receptor as a function of titrated receptor levels. This systems-level strategy has provided new insights into the biology of the EGFRvIII receptor and has identified the c-Met receptor as a novel target for the treatment of EGFRvIII expressing tumors.

### Materials and Methods:

### Sample Preparation, Peptide IP and Mass Spectrometry

U87MG cells expressing titrated levels of EGFRvIII were maintained in DMEM medium supplemented with 10% FBS. 1.5 million cells per 10 cm plate were washed with PBS and incubated for 24 hours in serum-free media. Cells were lysed with 8 M urea supplemented with 1 mM sodium orthovanadate (Sigma-Aldrich). For each biological replicate, three 10 cm plates were pooled together. The samples were then further processed and labeled with the iTRAQ reagent as previously described (Zhang et al. (2005) Mol Cell Proteom 4:1240-1250). Peptide immunoprecipitation was performed as previously described (Zhang et al. (2005) Mol Cell Proteom 4:1240-1250), with the following exceptions: 10 μg of protein G Plus-agarose beads (Calbiochem) were incubated with 12 μg of anti-phosphotyrosine antibody (pTyr100 (Cell Signaling Technology) in 200 μl of IP buffer (100 mM Tris, 100 mM NaCl, 1% NP-40, pH 7.4) for 8 hr at 4° C. Immobilized metal affinity chromatography (IMAC) was performed as previously described to remove non-specific non-phosphorylated peptides and eluted phosphopeptides were analyzed by ESI LC-MS/MS on a QqT of (QSTAR XL Pro, Applied Biosystems) operated in IDA mode, as previously described (Zhang et al. (2005) Mol Cell Proteom 4:1240-1250).

### Phosphopeptide Sequencing, Quantification and Clustering

MS/MS spectra were extracted and searched against human protein database (NCBI) using ProQuant (Applied Biosystems) as recommended by the manufacturer. Phosphorylation sites and peptide sequence assignments contained in ProQuant search results were validated by manual confirmation from raw MS/MS data. Peak areas for each of four signature peaks (m/z: 114, 115, 116, 117, respectively) were obtained from ProQuant and corrected for isotopic overlap. Peak areas were normalized with values from the peak areas of nonphosphorylated peptides in supernatant of the immunoprecipitation. Each condition was normalized against the U87H cell line to obtain fold changes across all 4 conditions. Final normalized data sets were loaded into Spotfire and the self-organizing map algorithm was used to cluster phosphorylation sites.

### Immunoblotting Analysis

For whole-cell extracts, cells were lysed in lysis buffer (20 mmol/L Tris-HCl, 150 mmol/L NaCl, 1 mmol/L EDTA, 1% Triton X-100, 2.5 mmol/L sodium PPᵢ, 1 mmol/ [symbol] glycerophosphate) containing protease and phosphatase inhibitors after the indicated treatment. Protein samples were separated on either 7.5% or 10% SDS-polyacrylamide gels and transferred onto polyvinylidene difluoride membrane. Blots were developed with supersignal West Femto substrate (Pierce) after incubation with primary and secondary antibodies.

### Kinase Inhibitor Treatment

Cells were serum starved for 24 hours prior to being treated with the indicated dose of either AG1478 or SU11274 (Calbiochem) for 1 hour. Cells were then lysed as described above for either immunoblotting or mass spectrometric analysis.

### Cell Viability Assays

4,000 cells were seeded per well in a 96 well plate. 24 hours later, the cells were serum starved for 24 hours prior to addition of fresh serum free media containing AG1478, SU1174, PHA665752 or cisplatin at the indicated doses and combinations. After 48 hours (for cisplatin treatment) and 72 hours (for kinase inhibitor treatments), cell viability was measured using the WST-1 reagent (Roche Applied Sciences), following manufacturer's recommendations.

### Apoptosis Assay

10,000 cells were seeded per well in a 96 well plate. 24 hours later, the cells were serum starved for 24 hours prior to addition of fresh serum free media containing AG1478, SU1174 at the indicated dose and combinations. After 24 hours of drug treatment, caspase 3/7 activity was measured using Apo-ONE Homogeneous Caspase-3/7 Assay (Promega), following the manufacturer's recommendations.

### Flow Cytometry

To enrich for U373 cells expressing inducible EGFRvIII-IRES-GFP or DK-IRES-GFP, cells were grown in the absence of dox and sorted for GFP expression using a FACStar (Becton Dickinson, San Jose, Calif.). For U87MG cells expressing various levels of EGFRvIII, a bulk population of cells was prepared by retroviral transduction with pLERNL and stained as described (Nishikawa et al. (1994) Proc Natl Acad Sci USA 91, 7727-7731) with anti-EGFR monoclonal antibody Ab-1 (clone 528; Oncogene Science, Cambridge, Mass.), followed by fluorescein isothiocyanate-conjugated goat anti-mouse Ig antibody (PharMingen, Minneapolis, Minn.) and sorted for low, medium, high, and superhigh receptor amounts. For this procedure, U87-EGFRvIII cells engineered previously and determined to express 2×10⁶ receptors per cell were used as a gating control (Nishikawa et al. (1994) Proc Natl Acad Sci USA 91, 7727-7731).

### HGF Elisa

Cells were serum-starved for 24 h before removal of media for measurement. Secreted HGF levels were measured using HGF Elisa kit (BioSource International, Camarillo, Calif.) according to the manufacturer's recommendations. After removal of media, cells were counted, and all HGF measurements were normalized to cell number.

### Anti-HGF Treatment

U87H cells were serum-starved for 24 h before treatment with either 5 μg/ml anti-HGF (R&D Systems, Minneapolis, Minn.) or 5 μg/ml control IgG (Sigma-Aldrich, St. Louis, Mo.) for 30 min. As a positive control, U87H cells were stimulated with 50 ng/ml HGF (R&D Systems) for 5 min after 30-min treatment with either anti-HGF or control IgG.

### Cell Culture, Retrovirus Infection, and Transfection

The human glioblastoma cell lines, U87MG and U373MG, and their engineered derivatives were cultured in DMEM with 10% FBS/2 mM glutamine/100 units/ml penicillin/100 mg/ml streptomycin in 95% air/5% CO₂ atmosphere at 37° C. U87MG cells expressing EGFRvIII or DK cells were selected in 400 μg/ml G418 and maintained, as described (Nishikawa et al. (1994) Proc Natl Acad Sci USA 91, 7727-7731). For expression of tetracycline-regulated EGFRvIII and DK, U373 glioma cells were transfected with pRev-tet-off (Invitrogen, Carlsbad, Calif.) by the calcium phosphate method (Furnari et al. (1998) Cancer Res 58:5002-5008) and selected in 400 μg/ml G418. Individual tetracyclin-controlled transactivator (tTA) expressing clones were analyzed for GFP expression, as expressed from transiently transfected pTRE-GFP, in the presence and absence of 1 μg/ml doxycycline (dox). A clone (c.16) demonstrating robust expression of GFP in the absence of dox was subsequently cotransfected with pBABE-puro and pTRE-EGFRvIII-IRES-GFP or pTRE-DK-IRES-GFP, and stable populations were obtained by selection in 1 μg/ml puromycin. Induction of EGFRvIII-IRES-GFP and DK-IRES-GFP was achieved upon growth in dox-free media.

### Xenografts

Cells (1×10⁶) were suspended in 0.1 ml of PBS and injected into the right flanks of nude mice. Tumor volumes were defined as (longest diameter)×(shortest diameter)²×0.5. All of the procedures were approved by the animal care and use committee of the University of California at San Diego.

### Experimental Results:

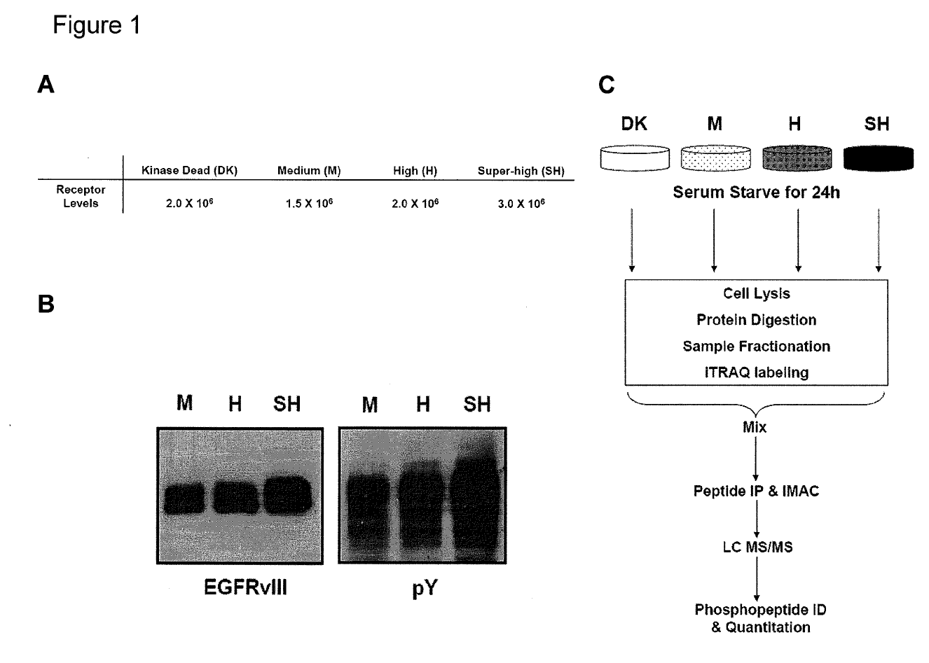

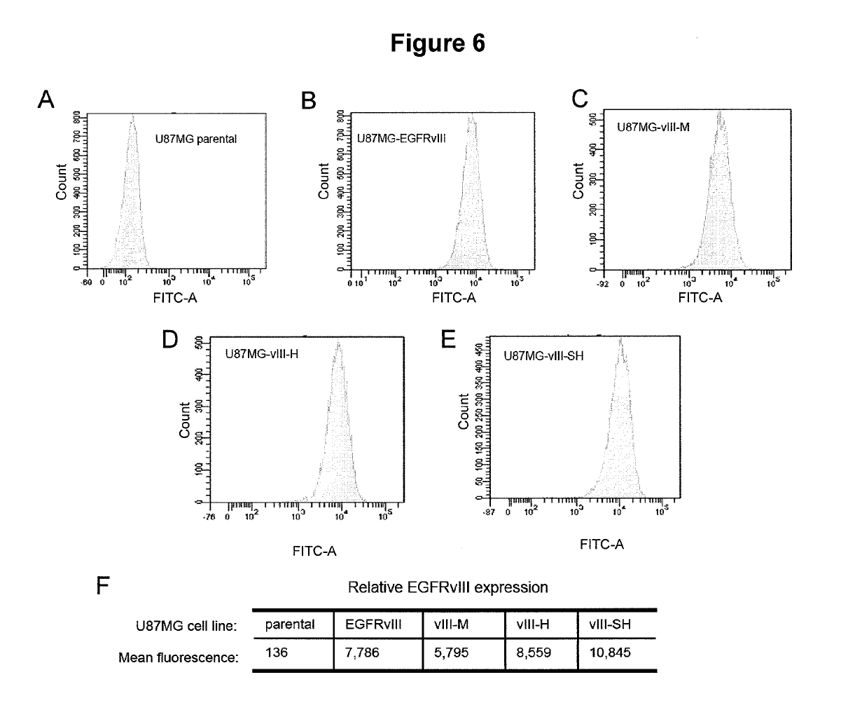

A mass spectrometric-based strategy was developed to identify and quantify tyrosine phosphorylation sites on cellular signaling proteins. In order to investigate the effect of EGFRvIII receptor load on phosphotyrosine-mediated cellular networks, this methodology was used to study U87MG glioblastoma cell lines expressing differential levels of EGFRvIII. The cell line has been transfected to express EGFRvIII and sorted into three populations expressing titrated receptor levels (listed in FIG. 1A). Western blot and FACS analysis confirm the expression levels of EGFRvIII as well as relative levels of tyrosine phosphorylation across the 3 cell lines (FIG. 1B and FIG. 6). Cells were serum-starved for 36 h, lysed, and probed for EGFRvIII or phosphotyrosine levels. A previously derived U87MG cell line expressing 2 million copies of a kinase dead version of the EGFRvIII receptor was used as a control. FIG. 6 shows EGFRvIII levels expressed in engineered U87MG cells. FIGS. 6A-E illustrate relative levels of membrane-expressed receptors in different cell lines as determined by FITC-conjugated antibody staining fluorescence intensity, and FIG. 6F summarizes the data. Fluorescence for U87MG parental cells was arbitrarily set to 100. U87MG-EGFRvIII correspond to cells previously characterized (Nishikawa et al. (1994) Proc Natl Acad Sci USA 91, 7727-7731).

As outlined in FIG. 1C, stable isotope labeled phosphotyrosine peptides were immunoprecipitated from the 4 cell lines after 24 hours serum starvation. These conditions were chosen in order to study the constitutive signaling pathways downstream of the EGFRvIII receptor. Following IMAC purification of the immunoprecipitated samples, liquid chromatography MS/MS analysis was performed to generate quantitative phosphorylation profiles for 99 phosphorylation sites on 69 proteins across the 4 cell lines. Two biological replicates were performed with an average SD of 15% for phosphotyrosine peptides that appear on both analyses.

### Quantitative Effects of Titrated EGFRvIII Levels on Receptor Phosphorylation and Major Downstream Signaling Pathways

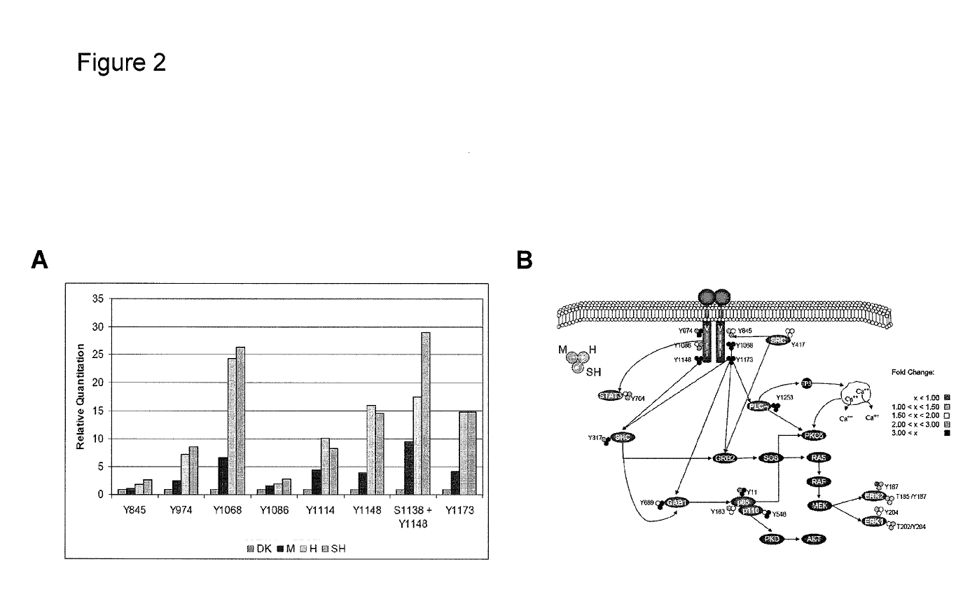

8 phosphorylation sites were identified and quantified on EGFRvIII (FIG. 2A). Phosphorylation levels are normalized relative to that of the DK cell line. Strikingly, each of the phosphorylation sites on EGFRvIII seems to be differentially phosphorylated as a function of increasing EGFRvIII receptor levels. Analysis of the phosphorylation profiles of the known autophosphorylation sites of EGFRvIII, Y1068, Y1148 and Y1173 revealed that the phosphorylation levels of these sites were not proportional to EGFRvIII receptor levels. A threshold receptor level of 2 million EGFRvIII receptors was required in order to mediate autophosphorylation on the receptor (15-25 fold activation). Below this threshold, less than 7 fold activation on these phosphorylation sites was observed. Conversely, there seems to be a saturating receptor level at which increasing receptor levels above 3 million copies does not seem to increase the autophosphorylation levels. This may be due to limiting amounts of downstream signaling proteins in the cell or the presence of negative feedback mechanisms regulating receptor autophosphorylation.

Mapping the data to the canonical signaling cascades downstream of wild-type EGFR (FIG. 2B) showed that EGFRvIII favors the utilization of different downstream pathways compared to wild-type EGFR. The treatment of wild-type EGFR expressing human mammary epithelial cells with exogenous EGF was demonstrated to led to a dramatic increase in the active form of Erk1, Erk 2 and STAT3 within 5 minutes of stimulation. In contrast, increasing EGFRvIII receptor load had little effect on the phosphorylation levels of these proteins. While a temporal analysis of wild-type EGFR signaling indicates that activation of this receptor only leads to a modest increase in the tyrosine phosphorylation levels on PI3K and its upstream adaptor protein GAB1, titrating EGFRvIII receptor levels dramatically increased the phosphorylation levels on sites of these 2 proteins by more than 3 fold, suggesting that the PI3K pathway is highly active in the EGFRvIII overexpressing cells. This data is consistent with previous reports that EGFRvIII activates the PI3K pathway which has been shown to be critical for promoting cell proliferation, survival and migration in GBM cell lines. According to aspects of the invention, preferential activation of this pathway by EGFRvIII (in addition to its constitutive activation) is related to its tumorigenic properties in vivo.

c-MET Receptor Tyrosine Kinase Activation is Highly Responsive to EGFRvIII Receptor Levels

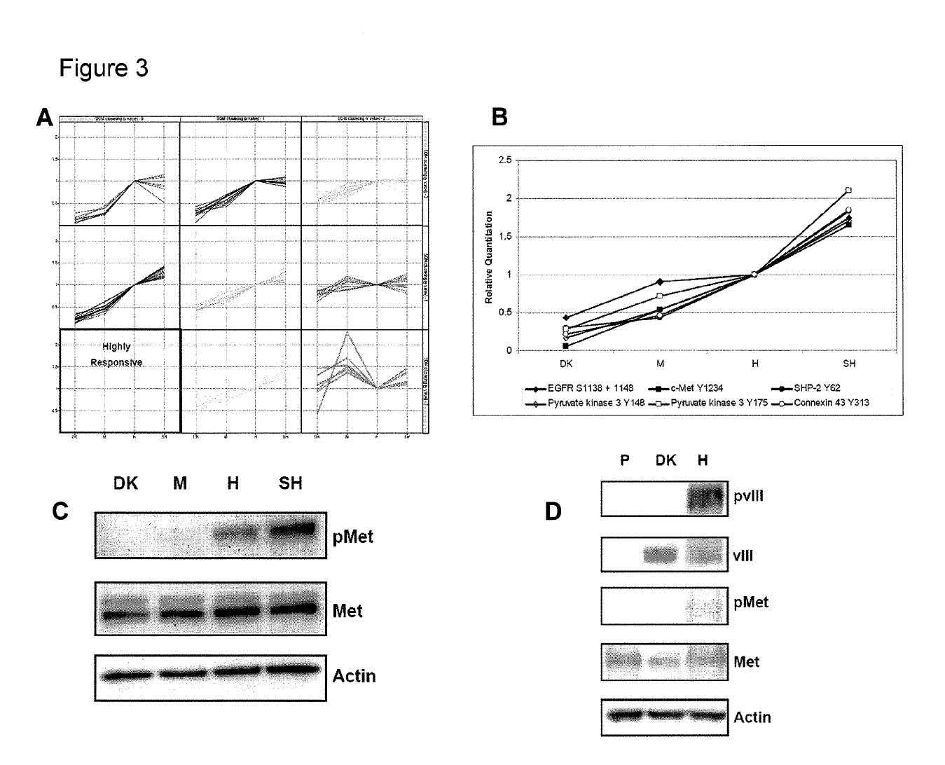

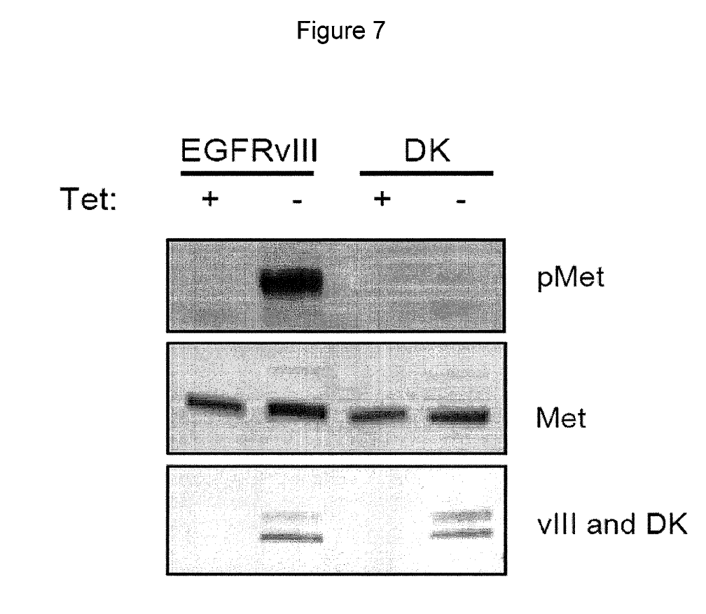

In order to identify clusters of tyrosine phosphorylation sites that exhibit similar profiles, the phosphoproteomic dataset was subjected to self-organizing map clustering (FIG. 3A). In FIG. 3A, each column within the matrix components represents the relative phosphorylation level in the -DK, -M, -H, and -SH U87MG cell lines normalized against the U87H cell line. Optimal SOM architecture was a 3×3 matrix, because smaller matrices tended to cluster dissimilar phosphorylation profiles. This analysis identified a cluster of phosphorylation sites that were highly responsive to EGFRvIII expression levels. Phosphorylation sites in this cluster showed dramatically increased levels as a function of increasing receptor dose and include Y1234 on the c-Met receptor tyrosine kinase (6 fold increase), an activating phosphorylation site in the catalytic loop of this receptor as well as Y62 on SHP-2 (10 fold increase), a protein tyrosine phosphatase which is a known downstream binding partner of the c-Met receptor (FIG. 3B). This activation of the c-Met receptor was confirmed by western blot analysis both in vitro across the 4 cell lines and also in vivo in xenografts (FIGS. 3C and 3D). In addition to the U87MG cell line, this EGFRvIII-mediated activation of the c-Met receptor was observed in tet-inducible EGFRvIII expressing U373MG glioblastoma cell lines (FIG. 7). FIG. 7 shows activation of c-Met receptor by EGFRvIII observed in U373MG cells through a Western blot of specific phosphorylation sites on the c-Met receptor (Y1230, Y1234, and Y1235) after 36-h serum starvation in tet-inducible U373MG cell lines expressing either EGFRvIII or the kinase-dead (DK) version of the EGFRvIII.

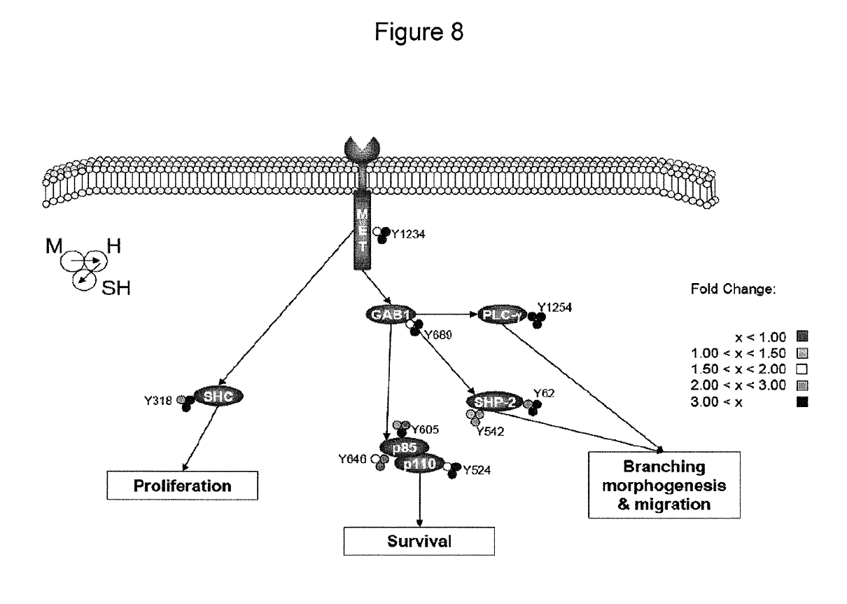

These observations indicate that the EGFRvIII receptor is constitutively activating the c-Met receptor pathway. Mapping the phosphoproteomic data to previously described pathways downstream of the c-Met receptor confirmed that many of the known downstream components of the c-Met receptor were activated at least 3 fold as a function of increasing EGFRvIII expression levels (FIG. 8). FIG. 8 is a schematic showing activation of the c-Met receptor network by EGFRvIII through visualization of the fold change in phosphorylation levels of the known canonical c-Met signaling cascades as a function of titrated EGFRvIII levels.

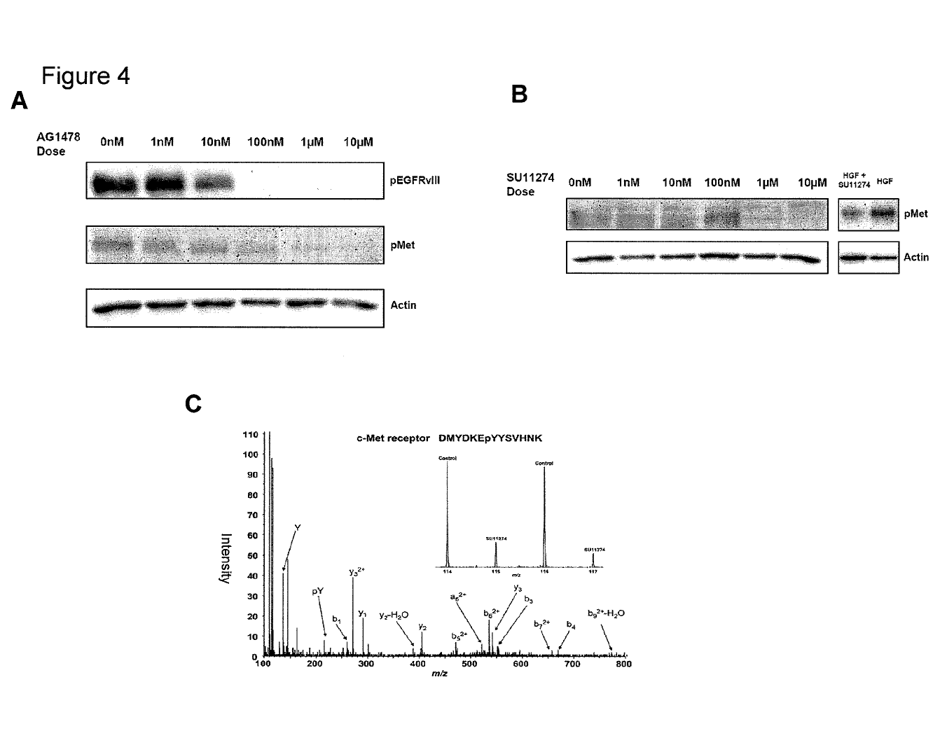

In order to demonstrate that c-Met receptor activation was a direct consequence of EGFRvIII receptor activation, U87H cells were treated with AG1478, an EGFRvIII kinase inhibitor. Western blot analysis revealed a dose-dependent decrease in EGFRvIII phosphorylation levels accompanied by a concomitant decrease in the phosphorylation status of c-Met (FIG. 4A).

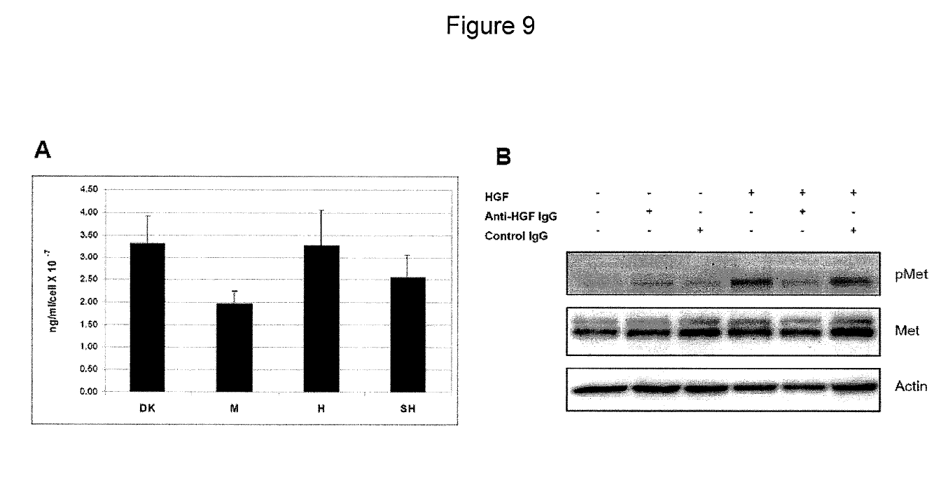

Since the U87MG cell line has previously been shown to express HGF, we sought to determine if this EGFRvIII-mediated c-Met activation was ligand dependent. Initial measurement of HGF secretion did not reveal any appreciable trends across the 4 cell lines when the values were normalized by the cell number (FIG. 9A). Treating the U87H cells with anti-HGF did not affect c-Met phosphorylation levels, suggesting that c-Met activation may not be ligand mediated but may involve some degree of direct signaling effects from EGFRvIII (FIG. 9B). FIG. 9B is a Western blot showing specific phosphorylation sites on the c-Met receptor (Y1230, Y1234, and Y1235) on the U87H cell line after 24-h serum starvation and treatment with either 5 μg/ml anti-HGF or goat control IgG; 50 ng/ml HGF treatment was used as a positive control. However, it is expected that ligand activation is probably also involved, and further experiments should better quantify this.

Combined Inhibition of the EGFRvIII and c-Met Receptors has Synergistic Effects on Cell Viability and Apoptosis

To determine the biological consequence of the c-Met activation, a c-Met specific kinase inhibitor, SU11274, was used. Treatment of U87H cells with an increasing dose of SU11274 led to a dose dependent decrease in c-Met receptor phosphorylation (FIG. 4B). This also was independently confirmed in biological duplicates using mass spectrometry (FIG. 4C). Two biological replicates were performed and peak areas for iTRAQ marker ions enable quantification of phosphorylation for each condition. Mass spectrometric analysis of SU11274 treated U87H cells indicate that this drug is exquisitely specific for the kinase activity of the c-Met receptor and does not affect EGFRvIII receptor tyrosine phosphorylation on multiple sites (data not shown).

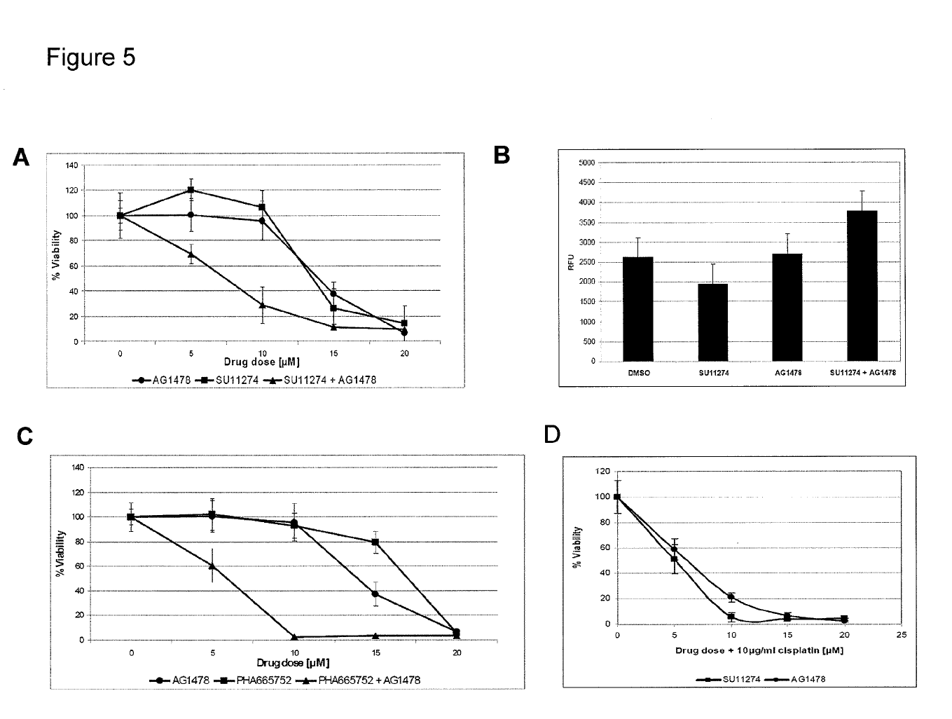

Due to the observed co-activation of EGFRvIII and c-Met receptors, co-treatment of EGFRvIII expressing cells with both EGFRvIII and c-Met kinase inhibitors may be expected to have an additive effect on cell viability and death. Treatment of U87H cells singly with either AG1478 or SU11274 followed a similar profile and only decreased cell viability at very high inhibitor doses. In contrast, combined dosing of SU11274 with a constant dose of 5 μM AG1478 led to a synergistic decrease in cell viability and an increase in cell death (FIGS. 5A and 5B). Viability was measured by using the metabolic dye WST-1. Combination treatment significantly enhanced cytotoxicity at 10 μM SU11274 (P<0.001). The concentration of drugs used was 10 μM SU11274, 10 μM AG1478, or a combination of 10 μM SU11274 and 5 μM AG1478. Combination treatment significantly enhanced apoptosis (P<0.01). This analysis also was performed with another c-Met inhibitor, PHA665752 and it was found to similarly synergistically sensitize the U87H cells upon co-treatment with AG1478 (FIG. 5C). The combination treatment significantly enhanced cytotoxicity at 10 μM PHA665752 (P<0.0001).

c-Met Kinase Inhibition Overcomes the Chemoresistance Properties Conferred by EGFRvIII

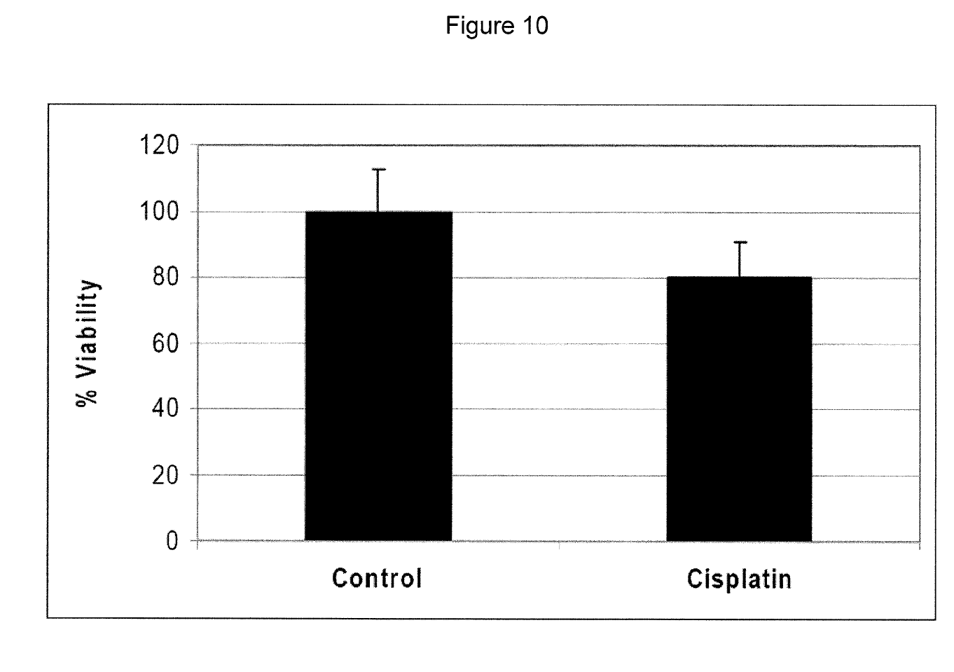

EGFRvIII confers chemoresistance to classical chemotherapeutics such as cisplatin through the modulation of BCL-XL and caspase 3, consequently, human glioblastoma xenografts expressing EGFRvIII were sensitized to cisplatin when co-treated with AG1478. Activation of the c-Met receptor has also previously been shown to confer cytoprotective properties to a wide variety of chemotherapeutics. According to aspects of the invention, the observed chemoresistance of EGFRvIII expressing tumors may in part be due to the constitutive activation of the c-Met receptor. In order to test this, the U87H cells were co-treated with increasing doses of SU11274 with a constant dose of 10 μg/ml of cisplatin. Compared to the cisplatin only treated control (FIG. 10), a dramatic decrease in cell viability was observed upon combination treatment (FIG. 5D). FIG. 10 is a graph showing that U87H cells are resistant to treatment with cisplatin. The response of U87H to 10 μg/ml of cisplatin treatment over 72 h after 24-h serum starvation is indicated. Viability was measured using the metabolic dye WST-1. This is similar to what is observed upon co-treatment of cisplatin with AG1478. This suggests that the c-Met receptor has a functional role in the chemoresistance of EGFRvIII positive tumors.

### Discussion:

Aspects of the invention relate to the first comprehensive analysis of the phosphotyrosine-mediated signaling pathways downstream of the EGFRvIII receptor. In this analysis, 101 phosphorylation sites on 69 proteins were identified and quantified, including 9 phosphorylation sites on EGFRvIII. While these phosphorylation sites on the EGFRvIII receptor may not be qualitatively different from those observed in wild-type EGFR, quantitative differences in the levels of phosphorylation at each individual site may have functional implications on resultant downstream signaling pathways and biological functions. Each of these phosphorylation sites was shown to be differentially phosphorylated as EGFRvIII receptor levels increase, suggesting that each site may be subject to differential regulation.

According to aspects of the invention, a threshold EGFRvIII receptor level is required to trigger autophosphorylation on the receptor. In addition, there seems to be a saturating receptor levels above which further increases in receptor dose does not have an influence on receptor autophosphorylation. This analysis provides the first systematic demonstration of the importance of oncogene dosage in the propagation of downstream cellular signaling pathways. Quantitative determination of such functional threshold limits for cancer genes represents a means to determine the relative order and dominance of oncogenes and their resultant cellular signaling pathways in human tumors containing multiple genetic lesions and provides for a fundamental understanding of the molecular basis of tumorigenicity in genetically heterogeneous human cancers.

Pathway analysis of phosphoproteomic dataset indicates that cells that overexpress EGFRvIII preferentially utilize the PI3K pathway over the MAP kinase and STAT3 pathways. This provides a mechanistic basis for the success of PI3K and mTOR small molecule inhibitors in combination with EGFR kinase inhibitors in the treatment of EGFRvIII expressing cells and xenografts. The ability of mass spectrometry-based network analysis to provide a mechanistic understanding of dysregulated signaling events in cancer highlights its utility in aiding in the selection of targeted therapies for use in the clinic.

Cluster analysis of the phosphoproteomic data reveals that the c-Met receptor is activated as a function of EGFRvIII receptor levels. This constitutive activation of the c-Met receptor by EGFRvIII is reminiscent of the constitutively active Tpr-Met fusion mutant of the c-Met receptor which may exhibit a more potent signaling potential that the transient receptor activation regulated by HGF ligand binding. According to the invention, it also has been determined that c-Met receptor activation does not require c-Met receptor overexpression as the crosstalk between the two receptors was observed in both the U87MG cell line which expresses low levels of c-Met and the U373MG cell line which overexpresses the c-Met receptor.

There are a wide variety of approaches to therapeutically regulate c-Met receptor activation. These include the use of anti-HGF monoclonal antibodies and c-Met small molecule kinase inhibitors. Preliminary data indicates that the constitutive c-Met activation in EGFRvIII overexpressing cells may involve some degree of direct signaling by the EGFRvIII receptor. However, ligand activation is also expected to occur, and inhibition of natural ligands are expected to be useful. EGFRvIII kinase inhibitors and c-Met kinase inhibitors synergistically act together to kill EGFRvIII expressing glioblastoma cells. These observations were made in the U87MG cell line which contains secondary genetic lesions commonly found to occur in human GBM patients, namely PTEN and Ink4A/Arf loss. PTEN is a tumor suppressor protein with both phosphoinositide and phosphotyrosine phosphatase activities and is commonly mutated in many advanced cancers including lung and prostate carcinomas.

Mellinghoff et al. have previously demonstrated that clinical response to EGFR inhibitors such as erlotinib and gefitinib in human glioblastoma patients was significantly associated with the co-expression of PTEN and EGFRvIII and recapitulated this observation in vitro in U87MG cell lines transfected to co-express both EGFRvIII and PTEN. Since PTEN mutation is seen in 30%-44% of high-grade gliomas, a large proportion of patients are refractory to EGFR kinase inhibitor therapy. The in vitro data suggests that co-treatment of EGFRvIII overexpressing tumors with both EGFR and c-Met kinase inhibitors may overcome this chemoresistance even in PTEN null tumors. Assaying for the expression of EGFRvIII and c-Met in human gliomas may guide the combined use of these inhibitors in the clinic.

Chemoresistance of diffused lesions in glioblastoma patients is a major reason for the almost 100% recurrence observed after surgical resection. It has been demonstrated that the EGFRvIII receptor confers drug resistance to classical chemotherapeutics such as cisplatin. This cytoprotective effect was also previously observed in glioblastoma cell lines upon activation of the c-Met receptor with HGF. Co-treatment of U87H cells with cisplatin and a c-Met kinase inhibitor led to a dose-dependent decrease in cell viability similar to what has previously been reported with EGFR kinase inhibitors. According to aspects of the invention, and without wishing to be bound by theory, the tumor-associated phenotypes previously solely attributed to the EGFRvIII receptor may in part be due its cross-activation of the c-Met receptor. The activation of multiple receptor tyrosine kinases by EGFRvIII may allow it to potentiate a multitude of additional tumorigenic properties. This may either be due to the independent activity of each activated receptor or an integrated signal arising from the combinatorial activation of multiple receptors. In addition to the activation of the c-Met receptor, the co-activation of the Axl and EphA2 receptors also was observed in this phosphoproteomic dataset. Accordingly, inhibition of multiple receptor tyrosine kinases may represent a therapeutic strategy to overcome the multifaceted clinical features seen in glioblastoma multiforme.

### Example 2

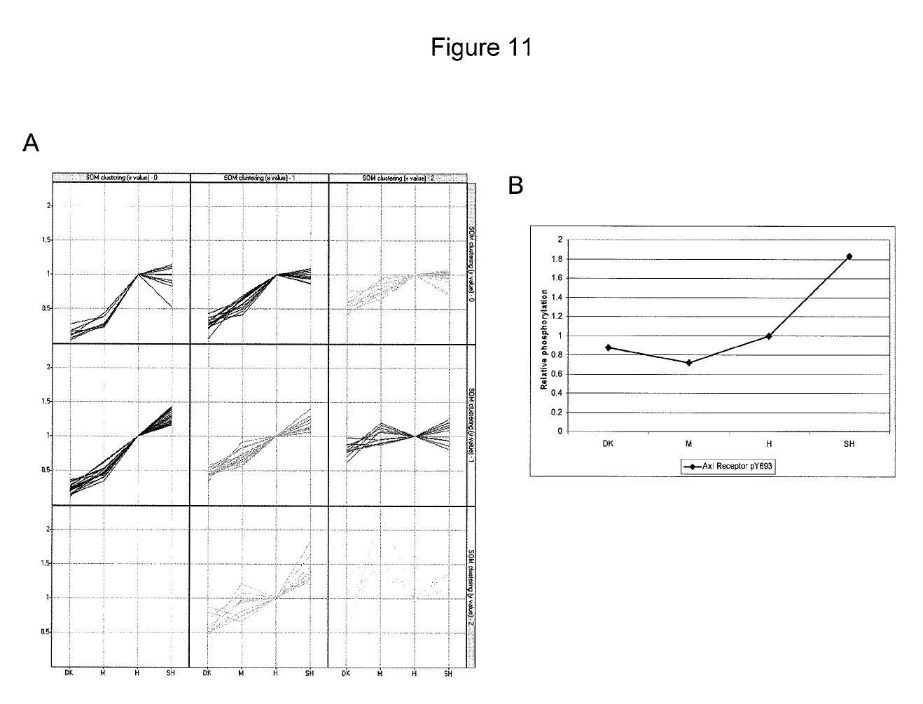

A mass spectrometry-based phosphoproteomic technique was used to investigate signaling networks downstream of the EGFRvIII oncogenic receptor in U87MG glioblastoma cells. U87MG cells expressing titrated levels of EGFRvIII were maintained in DMEM medium supplemented with 10% FBS. 1.5 million cells per 10 cm plate were seeded for 24 hours. Following this, cells were washed with PBS and incubated for 24 hours in serum-free media. Cells were lysed with 8 M urea supplemented with 1 mM sodium orthovanadate (Sigma-Aldrich). For each of the two biological replicates performed, lysates from three 10 cm plates were pooled together. The samples were then further processed and labeled with the iTRAQ reagent following manufacturer's recommendations. Peptide immunoprecipitation was performed as previously described (Zhang, Y., Wolf-Yadlin, A., Ross, P. L., Pappin, D. J., Rush, J., Lauffenburger, D. A. & White, F. M. (2005) Mol Cell Proteomics 4, 1240-50), with the following exceptions: 10 μg of protein G Plus-agarose beads were incubated with 12 μg of anti-phosphotyrosine antibody (pTyr100) in 200 μl of IP buffer (100 mM Tris, 100 mM NaCl, 1% NP-40, pH 7.4) for 8 hr at 4° C. Immobilized metal affinity chromatography (IMAC) was performed and eluted phosphopeptides were analyzed by ESI LC-MS/MS on a QqT of (QSTAR XL Pro, Applied Biosystems) as previously described (Zhang et al., 2005). FIG. 11 demonstrates the data obtained from the mass spectrometric analysis. Each column within the matrix components represent the relative phosphorylation level in the -DK, -M, -H, and -SH U87MG cell lines normalized against the U87H cell line. The cluster containing the Axl phosphorylation site is enlarged on the right. Axl phosphorylation increases in response to increased expression of EGFRvIII in U87MG cells, suggesting that the EGFRvIII receptor activates the Axl receptor.

### INCORPORATION BY REFERENCE

All of the scientific and patent publications referred to herein and in the attachment are incorporated herein by reference in their entirety. In the event of conflicting disclosures, the present detailed description is controlling.

1. A method for treating a cancer associated with constitutive EGFR signaling, the method comprising:
administering to a subject having a cancer that exhibits constitutive EGFR signaling a therapeutically effective amount of a composition that inhibits a c-Met signaling component.

2. (canceled)

3. The method of claim 1 wherein the cancer that exhibits constitutive EGFR signaling expresses EGFRvIII.

4. The method of claim 1 wherein the cancer is glioblastoma.

5. The method of claim 1 wherein the c-Met signaling component is c-Met.

6. The method of claim 1 wherein the c-Met signaling component is SHP-2.

7. The method of claim 1 wherein the c-Met signaling component is PLC-gamma.

8-9. (canceled)

10. The method of claim 1 wherein the composition that inhibits a c-Met signaling component is SU11274.

11-13. (canceled)

14. The method of claim 1 wherein the composition that inhibits a c-Met signaling component inhibits two or more c-Met signaling components.

15-16. (canceled)

17. A method for treating a cancer associated with constitutive EGFR signaling, the method comprising:
administering to a subject having a cancer that exhibits constitutive EGFR signaling a therapeutically effective amount of a composition that inhibits an Axl signaling component.

18. (canceled)

19. The method of claim 17 wherein the cancer that exhibits constitutive EGFR signaling expresses EGFRvIII.

20. The method of claim 17 wherein the cancer is glioblastoma.

21. The method of claim 17 wherein the Axl signaling component is Axl.

22. The method of claim 17 wherein the Axl signaling component is SHP-2.

23. The method of claim 17 wherein the Axl signaling component is PLC-gamma.

24-31. (canceled)

32. A method for determining whether a cancer patient should be treated with a composition that inhibits a c-Met signaling component, the method comprising:
(a) performing an assay to determine whether a patient has a cancer that exhibits constitutive EGFR signaling; and,
(b) identifying the patient as being a candidate for treatment with a composition that inhibits a c-Met signaling component if the patient has a cancer that expresses c-Met and exhibits constitutive EGFR signaling.

33. (canceled)

34. The method of claim 32 wherein the cancer that exhibits constitutive EGFR signaling expresses EGFRvIII.

35. The method of claim 32 wherein the cancer is glioblastoma.

36. The method of claim 32 wherein the c-Met signaling component is c-Met.

37. The method of claim 32 wherein the c-Met signaling component is SHP-2.

38. The method of claim 32 wherein the c-Met signaling component is PLC-gamma.

39-121. (canceled)
